(19) United States
(12) Patent Application Publication
(10) Pub. No.: US 2014/0207191 A1
(43) Pub. Date: Jul. 24, 2014

(54) System and Method for Performing Spinal Stabilization

(71) Applicant: Ezriel Kornel, Bedford Hills, NY (US)

(72) Inventor: Ezriel Kornel, Bedford Hills, NY (US)

(21) Appl. No.: 13/862,551

(22) Filed: Apr. 15, 2013

Related U.S. Application Data
(63) Continuation-in-part of application No. 11/650,096, filed on Jan. 5, 2007, now Pat. No. 8,486,077.

(51) Int. Cl. A61B 17/70; A61B 17/16

(52) U.S. Cl. CPC A61B 17/7068; A61B 17/1671; A61B 17/7074
USPC 606/248; 606/279

(57) ABSTRACT

A system and method for performing percutaneous spinal stabilization, comprising inserting a k-wire into skin and tissue proximal to the spine; drilling the k-wire into and through the spinous process, creating a narrow aperture therethrough; inserting one or more cannulas over the k-wire, through the skin and tissue and towards said bone, a second cannula configured to fit over the first; removing all but the outermost cannula, thereby creating a working channel through the skin and tissue, providing unobstructed access to the bone; inserting into the channel a screwdriver comprising a preferably flexible shaft long enough to extend through the cannula to the bone, and having releasably attached to the end a screw with external screw threads; and drilling one or more screws into and through the bone for securement to a cap secured to a hollow, oval, flat stabilizing plate positioned on the opposite side of the bone.

### RELATED APPLICATIONS

This application is a Continuation-in-Part application of, and claims priority to, pending U.S. Non-Provisional patent application Ser. No. 11/650,096, filed Jan. 5, 2007, the disclosure of which is incorporated by reference herein, and is also based on U.S. Pat. No. 8,097,021, which issued Jan. 17, 2012, itself based on Non-Provisional application Ser. No. 12/004,954, filed Dec. 21, 2007, which itself is based on Provisional Patent Application No. 60/876,946, filed Dec. 22, 2006.

### FIELD OF THE INVENTION

The present invention relates to a system and method for performing percutaneous spinous process fusion, and specifically the insertion of screws or screw-like holding devices (hereinafter collectively referred to as “screws”) by means of a drill or screwdriver-like device (hereinafter collectively referred to as “a drill”) into the spine through a working channel previously surgically provided therein, which will enable securement of a stabilizing plate, designed to connect to one or more vertebrae to produce a more stable condition.

### BACKGROUND OF THE INVENTION AND DISCLOSURE

Certain types of spine diseases are caused by painful relative motion of vertebrae of the spine. The pain and discomfort often warrant a procedure known as spinal fusion. Such abnormal and painful motion can be caused by proximity of discs, abrading of protective material between disks, abnormal slippage of the vertebrae and other degenerative spinal conditions. In addition, certain conditions involving instability of the spine, and certain types of fractures, infections, deformities or tumors may also warrant spinal fusion. Traditionally, an open surgical or invasive technique is used, according to which an incision is generally first made; tissue and muscle are separated; a portion of a bone is removed or decorticated; and a bone fusing substance, such as some combination of allograft bone (from a donor) or autograft bone (from the patient) is inserted between the decorticated areas of two vertebra. There are generally two types of spinal fusion: posterolateral fusion, which includes placement of a bone graft between the transverse processes in the back of the spine, and interbody fusion, which includes placement of a bone graft between the vertebrae in the area usually occupied by the intervertebral disc. A third technique is interspinous process stabilization and fusion.

The present invention relates to a novel system and method for performing percutaneous spinous stabilization. A stabilizing plate, which rests against the spinous process, can be percutaneously inserted into a patient and secured by use of a set of tools and surgical hardware, and by use of a delivery system which allows for the delivery of components in a relatively minimally invasive manner.

The present invention provides a system and method for inserting a holding mechanism into the spine for secure attachment to a stabilizing plate. In the preferred embodiment, a k-wire, or sterilized, sharpened, smooth stainless steel rod, is first inserted through the skin of a patent and into the spine. Once the k-wire has been inserted into the body, a first cannula is inserted over the k-wire and into the spine, thereby widening the bore-like or cylindrical opening formed in the skin and tissue by the k-wire insertion. After the first cannula has been inserted, a second cannula is inserted through the skin and into the spine, and it is slid or placed over the first cannula. The k-wire can be removed. This larger bore cannula follows the same path to the spine. Once the second cannula is in place, thereby again widening the opening in the skin and tissue and creating a clear, unobstructed working channel to the spine, the first cannula can be removed.

With the second cannula in place and a working channel provided, a screwdriver shaft (flexible or not) can be inserted into the channel of the cannula, through the opening in the skin and tissue, and extending toward the spine. The screwdriver preferably has a screw, staple. or other connecting means, secured to its end and projecting into the working channel and towards the spine.

A stabilizing plate, similar to that shown and described in U.S. Pat. No. 8,097,021, in the preferred form, a hollow center, oval-shape with a flat top and bottom surface and having curved outside walls on its ends, is inserted by means of a carrier clamp, which can also be introduced to the spine by means of a percutaneous channel or tube delivery system. The clamp may be one-armed or may include a set of opposed finger-gripping handles with finger holes, just like a pair of pliers, to facilitate manual manipulation. The clamp is hinged at a central pivot point. It has two arms and associated ends which are adapted and designed to accommodate, hold, and selectively release the stabilizing plate, held on the arm of the clamp.

Using the clamp, the stabilizing plate can be maneuvered into place through a percutaneous delivery system. Once in place, the drill or screwdriver with screw or staple at its distal end can be inserted into the cannula and through the working channel. The screw can be drilled through the spine utilizing a drill (either manual or mechanical). The screw will be driven into the bone, entering on one end and held to the bone by the screw's proximal end or head and protruding out of an opposing side of the bone, that opposing or distal end of the screw being adjacent to the stabilizing plate and intended to be secured thereto.

In the preferred embodiment, the screw can then be turned until its outer screw thread catches or engages to an equivalent inner screw thread of a cap-like element secured to or within the stabilizing plate or on the other side of the plate. The screw can be turned and tightened until it holds the cap of the stabilizing plate securely so as to prevent any movement of the plate. The plate will be held against the bore. In an alternate embodiment, the screw can pass through the cap of the stabilizing plate and attach to a nut secured to the opposing side of the stabilizing plate. Upon passing through the stabilizing plate and being twisted into the screw thread of the nut, the screw will tighten and securely attach to the nut, thereby pulling the plate against the bone and preventing the stabilizing plate from moving, once positioned on the bone(s) of the spine.

A second screw can be inserted, using the same method, into a second bone location (same or different bone) of the spine which can be similarly screwed into a cap or nut attached to or holding the stabilizing plate. When two or more screws are inserted by this method, through multiple bone locations in the spine, and secured to the stabilizing plate, the plate will be immovably positioned adjacent to bone(s) of the spine.

### DESCRIPTION OF PRIOR ART

Certain types of minimally invasive spine procedures have become known. Boehm et al., U.S. Pat. No. 6,666,891 teaches an interbody spine fusion method, according to which a disk of the spine is removed, and bone matrix is passed through a dilator to encourage fusion at the disc space. Also, Helm et al., US Patent Application Publication Number 2005/0038514 discloses a spine fusion method and system, according to which the disc is removed and fusion is encouraged at the disc cavity. Also known is an X-tube procedure for interbody spinal fusion in which a disk is removed, and a sextant is used to attach rods and screws. These references and the X-tube procedure however, involve removal of a disk of the spine and are not directed to performing a spinal fusion at a transverse process with relative less surgical invasiveness.

Further, Boehm et al., US Patent Application Publication Number 2005/0203529 describes a minimally invasive method for spinal fusion using a bone graft capsule for facilitating the fusion, such that a portion of the transverse processes of vertebrae is first cut and a string of capsules strung on a bioabsorbable string is introduced between the transverse processes by means of a device inserted into the body via a separate incision, which device grasps the string.

However, this technique seems to require multiple incisions in the patient's body and requires cumbersome application of the bone matrix or bone fusion substance. Further, these references do not disclose pushing the bone fusion substance via a delivery tube to the decorticated transverse processes or to other areas of interest.

The present invention is directed to a mechanism set, screw or staple, which pass through a bone and secure to a stabilization plate held against the bone, by the leading tip of the screw or staple mechanically coupling to a cap or nut held against the plate.

### SUMMARY OF THE INVENTION

The present invention discloses a method and system for performing percutaneous spinal fusion and/or stabilization. The method comprises the steps of first inserting a k-wire (with pointy tip) into the skin near to a bone in the spine and directing it towards the base of the spinous process; drilling, by means of a manual or motorized drill, the k-wire into and through the bone, thereby creating a narrow aperture through the spinous process; inserting one or more cannulas, and preferably two, with one fitting within the channel of the other, over the k-wire and through the skin and tissue and towards the bone. Once the k-wire and any internal cannulas are removed, a working channel is created in the remaining cannula through the skin providing unobstructed access to the bone. A screwdriver or drill can be inserted into the channel, the screwdriver comprising a shaft long enough to extend through the cannula to the bone. The screwdriver or drill preferably has, attached on the end which enters the skin, a screw or drill bit, which is then drilled through the bone and secured to a cap or nut associated with a stabilizing plate positioned on the opposite side of the spinous process. The plate, preferably, is a longitudinal hollow oval washer-like device with a thin wall. This process can be done one or more times so as to fuse multiple bones together for bone stabilization.

### BRIEF DESCRIPTION OF THE DRAWINGS

FIG. 1A discloses a multitude of tools and hardware which can be used with the present invention, a system and method of performing spinal stabilization and/or fusion;

FIG. 1B discloses a stabilizing plate which can be used with the present invention, as well as two proposed mechanisms attached on one side of the stabilizing plate for securement of the plate to the spine when a screw or staple is captured thereby;

FIG. 2 discloses a carrier tool which can be used to introduce a stabilizing plate in place on the spine (the plate can have the caps, staples, or nuts screwed to one side of the plate);

FIG. 3 discloses the first three steps of the method of performing spinal fusion, consistent with the present invention, as disclosed by the preferred embodiment of the present invention. Step 1 shows a cross-sectional view of the spinal column, the skin, and the introduction of the k-wire. Step 2 shows the use of a drill to pass through the bone, through which the lead end of the screw will be passed into the cap of the plate. Step 3 shows the cannula forming a working channel over the k-wire, extending to the bone;

FIG. 4 discloses the next three steps (Step 4, Step 5A and 5B) of the method of performing spinal stabilization, which would directly follow the steps disclosed in FIG. 3 in the preferred embodiment of the present invention. Step 4 shows the introduction of the wider bored cannula, over the k-wire and first cannula; Steps 5A and 5B show the use of a drill and/or screwdriver, respectively, (or mechanical power driver) and a screw passing through the bone and to the capturing plate;

FIG. 5 discloses the final two steps of the method of performing spinal stabilization, which would directly follow the steps disclosed in FIG. 4 in the preferred embodiment of the present invention. Here, Step 6 shows the screw being captured by the cap end of the plate while Step 7 sows the plate and screw held thereby secured to bone;

FIG. 6 discloses an alternate view of the first step of the method for performing spinal stabilization, as seen in FIG. 3, from the perspective of the cross-sectional side view of the spinous process and showing the use of k-wire into two adjacent bones;

FIG. 7 discloses the stabilizing plate as it would be placed using the carrier plate tool, shown in FIG. 2 to be placed on the spine and also shows the caps to be captured by the screw;

FIG. 8 discloses an alternate view of the third step of the method for performing spinal stabilization, as seen in FIG. 3, from the perspective of the side of the spinous processes ad shows the plate extending to two adjacent bones;

FIG. 9 discloses the stabilizing plate as it appears after being secured to the spine of a patient using the system and method of the present invention and stabilizing adjacent bones; and

FIG. 10 discloses the spine and adjacent bones with stabilizing plate secured to the spine thereto (or far side of the bones), as seen in FIG. 9.

### DETAILED DESCRIPTION OF THE DRAWINGS AND THE PREFERRED EMBODIMENTS

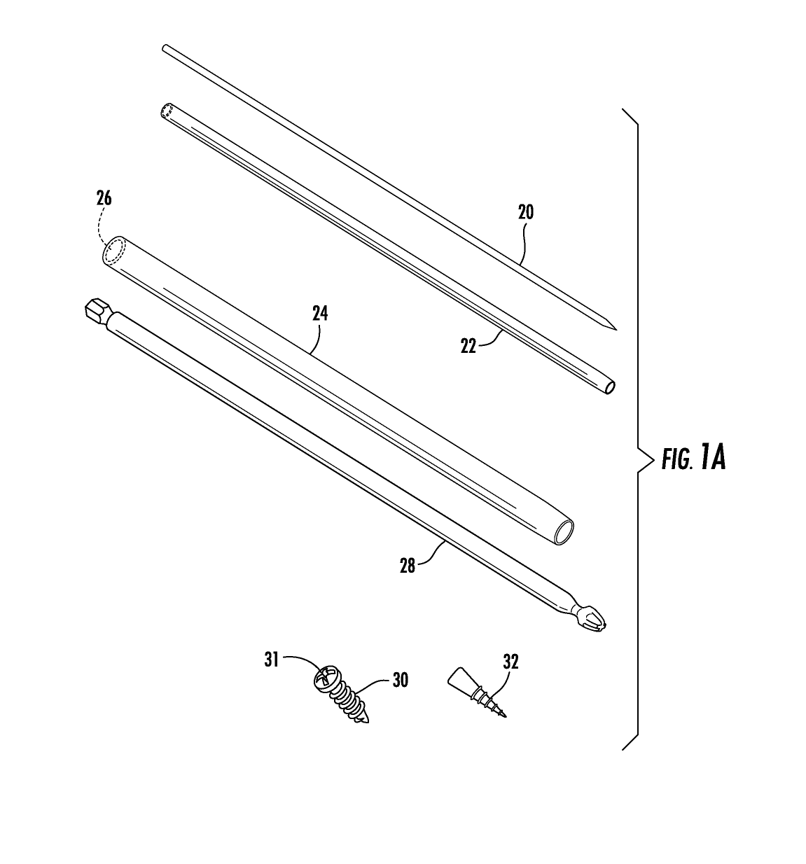

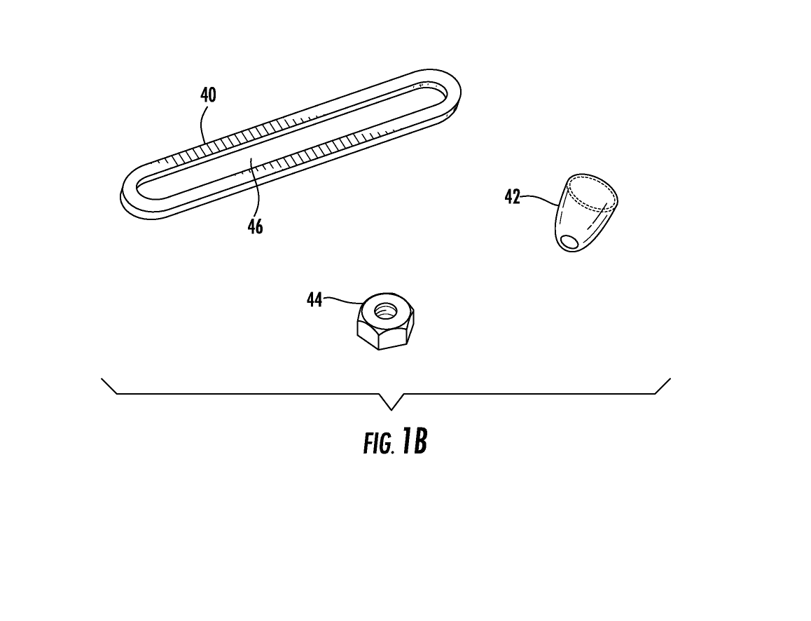

Description will now be given of the invention with reference to the attached FIGS. 1-10. It should be understood that these figures are exemplary in nature and in no way serve to limit the scope of the invention as the invention will be defined by the claims, as interpreted by the Courts in an issued US patent.

FIG. 1A discloses the various tool implementations which can be utilized by the preferred embodiment of the present invention. A k-wire 20 (with leading piercing tip) is preferably inserted into the skin of a patient and directed down towards the spine and more precisely to vertebrae on which spinal fusion and stabilization is desired to be performed. All conventional methods of surgery are intended to be followed, e.g., manner of location of the bones, anesthetics, hygiene, typical out-patient and/or in-hospital prep, etc. A first cannula 22 is preferably inserted over the k-wire after the k-wire has been placed within the skin, tissue, and spine of a patient, the first cannula serving to widen the aperture in the skin caused by the smaller relative diameter of the k-wire. A second cannula 24 is then preferably placed and slid over the first cannula 22, in the same manner as the first cannula 22 was placed over the k-wire 20, in order to even further widen the bore/channel in the skin and tissue. In alternate embodiments of the present invention, a second cannula is not required. However, it is utilized in the preferred embodiment. The second cannula 24, with a longitudinal bore or opening through its center (in which the k-wire 20 and first cannula 22 have passed, creates a wide diameter working channel 26 through the skin, muscle, and tissue and towards the spine (bones) of a patient to provide unobstructed access to the bones intended to be stabilized.

Once the second cannula 24 is in place, the k-wire 20 and first cannula 22 can be removed from the channel 26, leaving a longitudinal opening down the center of the second cannula 24. The K-wire may be left in place and a drill with a hollow center in the bit or screwdriver may be placed over the K-wire. Once the opening is provided by the second cannula, a drill or screwdriver 28 is preferably placed through the working channel 26 and towards the spine. The screwdriver or drill may have, and preferably has, a flexible shaft. A hollow screw 30 or staple 32 containing outside gripping ridges can be securably yet releasably attached to the end of the drill or screwdriver 28. The screwdriver 28 with screw 30 (or drill with staple) can then be passed through the working channel 26 of second cannula 24 over the K-wire and extending towards the bone(s) of the spine. Alternatively, the K-wire could be removed and the screwdriver/drill then inserted through the cannula and the screw placed as desired.

FIG. 1B discloses a stabilizing plate 40 which can be used by the present invention. It is similar to that shown in U.S. Pat. No. 8,097,021. The stabilizing plate 40, in the preferred form, is a hollow, flat, oval shape, opposed top and bottom flat surfaces, with curved outside walls on its ends. It is a washer-like device. The stabilizing plate 40 is provided with an open, oval-shaped center 46 through which the tips of screws 30 or staples 32 project and secure into caps 42 or nuts 44 securely attached thereon. The screws 30 will preferably pass through the spine and connect to the inside of one or more caps 42 or to internal screw threads of nuts 44, the caps or nuts being secured to or against the far side of the stabilizing plate 40. The external screw threads of the staples or screws 30 mate with corresponding internal screw threads on the caps 42 or nuts 44. When the stabilizing plate 40 is inserted and secured to the far side of the bones of the spine, it will be held in place by the screws 30 or staples 32 attaching to either cap 42 held within the center or open area 46 of stabilizing plate 40 or nut 44, which will preferably be located on the side of the stabilizing plate 40 opposite the side adjacent to the spinous process. The nuts or caps are preferably pre-welded or secured to the plate. Alternatively, a plate may be placed on both sides of the spinous process with the screws/staples passing through both plates and with the screws/staples securing the plates in position.

On one side of the spinal processes of the patient will be the heads of the screws 30 or staples 32 (alternatively a plate and screw/staple heads can be on both sides) while on the other side of the processes of the patient, the stabilizing plate 40 and the locking caps 42 or nuts 44 will be located, thereby immobilizing the spinous processes by the long, flat plate being pushed against it. The inside of the locking caps 42 in the preferred embodiment would be a tapered conical cavity, adapted to mate with and frictionally hold onto the pointed tip of the screw 30 so that when secured, the locking cap 42 will not accidentally be dislodged. The flat bottom wall of the stabilizing plate 40 serves to provide a surface against the processes which, when the screws 30 are secured in the caps 42 of the stabilizing plate 40, pressure maintains the processes under compression and the bone is immobilized. The stabilizing plate 40 rests against the spinous processes on the opposite side from the screws' heads 31 and their initial points of entry into the vertebrae. Alternatively, a plate rests on both sides of the spinous processes and screws are used to hold the plates against the spinous process to stabilize the same and one another.

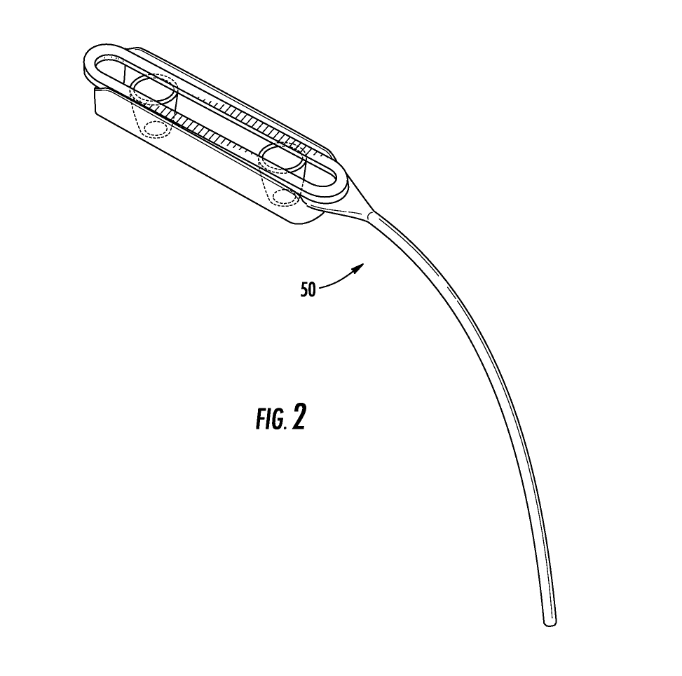

FIG. 2 discloses a carrier or tool 50 which can be used in the present invention to insert and hold the stabilizing plate 40 while the screws 30 or staples are inserted therein to the caps or nuts. The carrier 50 can be introduced percutaneously via a percutaneous tube delivery system. The basics of that inventive tubular delivery system is fully described and shown in non-provisional U.S. patent application Ser. No. 11/650,096 filed Jan. 5, 2007, the disclosure of which is specifically incorporated by reference herein. In brief summary, an incision is first made in the body after suitable anesthetic takes “hold” and a trocar inserted to create access to the vertebrae of interest. A trocar has a sharp tip and is typically a solid object like a rod. Preferably it is curved. The trocar may be embodied as part of a cannula, such as a cannular trocar. Then, a dilator is inserted over the trocar and a suitable wider channel is developed. The trocar is then removed. The dilator is typically used to create a wider channel than the one made by the trocar. The dilator typically has a hollow center bore so that it slides over the in-place trocar before the trocar is removed. A delivery tube, preferably u-shaped in cross-section, is inserted or threaded over the dilator. With the delivery tube in place, the dilator can then be removed by removing it through the delivery tube. The carrier 50 can then be inserted through the delivery tube with the plate removably secured thereto, at the precise desired location.

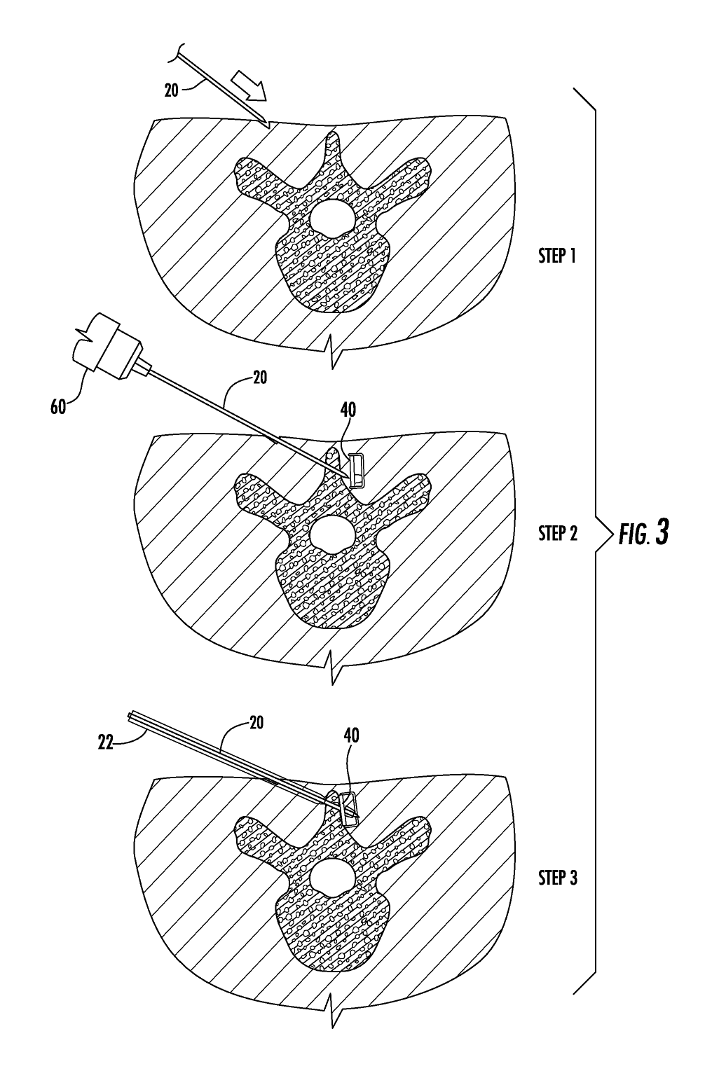

FIG. 3 discloses the first three steps of the method for performing spinal stabilization described by the preferred embodiment of the present invention. As seen by Step 1, a distally, pointed end, k-wire 20 is inserted into a small incision in the skin and directed to the spinous process. Step 2 discloses the use of a drill, whether manual or power-controlled, to push the k-wire 20 further through the skin and towards and through the spinous process, towards the stabilizing plate 40 having been inserted by the method and tool discussed above. Once the k-wire 20 or drilling point has been drilled through the spinous process toward the stabilizing plate 40 (located on the opposite side thereof), first cannula 22 can be inserted over the k-wire, with the cannula passing over the k-wire. The cannula has a longitudinal central opening. This is shown in Step 3. The first cannula 22 creates a wider working channel or bore in the skin than the k-wire, thus creating a greater unobstructed path to the bone of the spine.

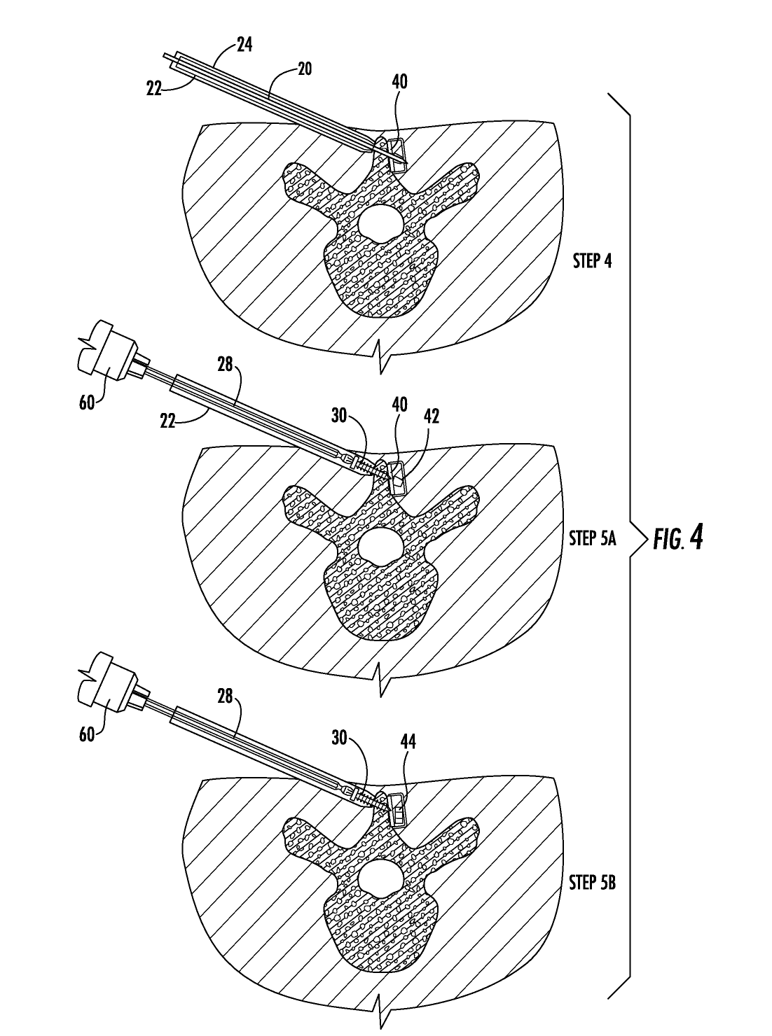

FIG. 4 discloses the next steps of the method disclosed by the preferred embodiment of the present invention. In step 4, a second cannula 24 is preferably inserted through the aperture in the skin and over the first cannula 22, causing the channel 26 in the second cannula 24 to pass over the k-wire 20 and first cannula 22. Once the second cannula 24 has been inserted through the skin and towards the spine, the k-wire 20 and first cannula 22 are preferably removed, leaving a widened working channel or aperture through the second cannula, with unobstructed access to the spinous processes.

Once the channel 26 has been developed, the screwdriver 28 with a screw 30 removably secured on one end can be inserted into and through the channel 26 towards the bone. The screw 30 can then be drilled, using drill 60, through the spinous process. This is shown in Steps 5A and 5B (electric and mechanical drills/screwdrivers). The sharp tip end of the screw 30 (or a flat end) will preferably pierce through the spinous process and mate with a cap 42 (or nut 44), attached to the hollow oval portion of the stabilizing plate 40, on the side opposite of the spinous process. The screw 30 will attach by means of its external screw threads, being twisted and tightened until it is held firmly and securely in place within the internal screw threads of the cap 42. Or the external threads of the screw will be captured by nut 44. The proximal or driving head of the screw 31 will rest securely on the side of the spinous process into which the screw was inserted, and the stabilizing plate 40, with cap 42 or nut 44 attached thereto and end of screw 30 secured therein, will sit securely on the opposite side of the spinous process. Alternatively the screw head may rest against the proximal plate. This can be done for a multitude of vertebrae in order to fuse or stabilize more than one together.

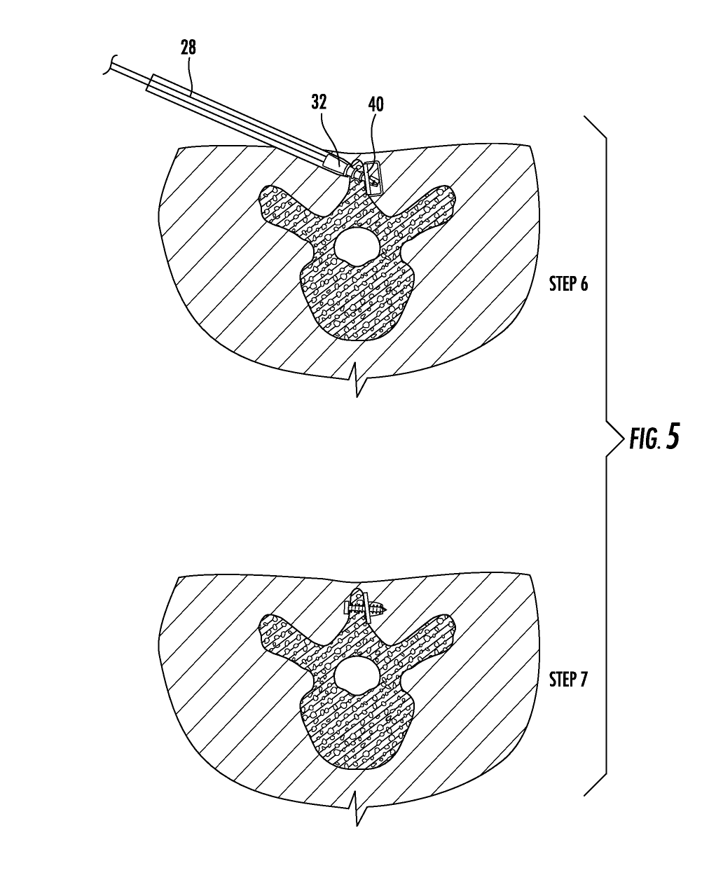

FIG. 5 discloses an alternate embodiment of the present invention, showing in Step 6, the use of a staple with external ridges 32 as opposed to a screw 30 for connection to the stabilizing plate. Step 7 discloses the staple 32 or screw 30 as it would appear once connected to a cap 42 or nut 44 on the stabilizing plate 40, after the drill 28 and second cannula 24 have been removed from the body of the patient.

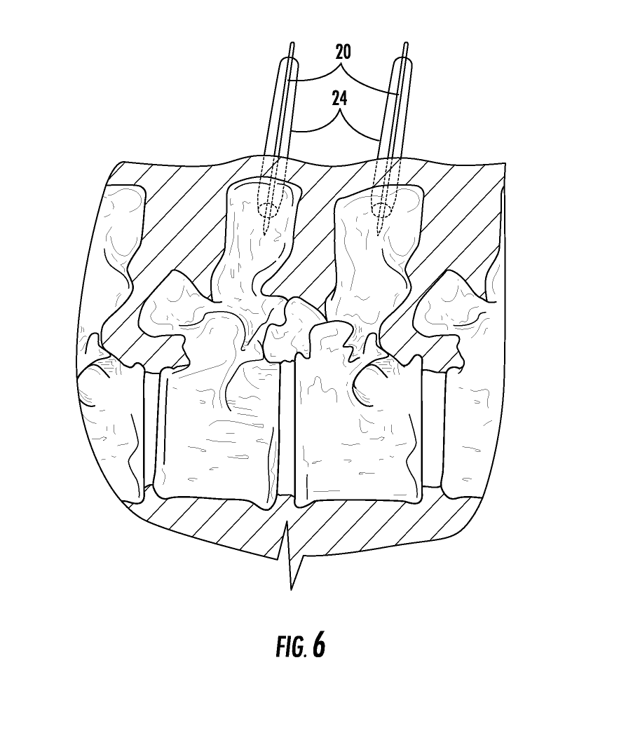

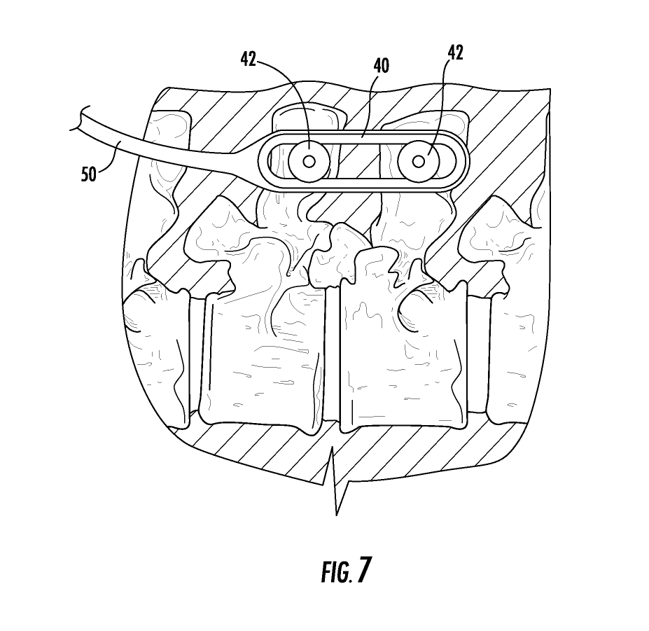

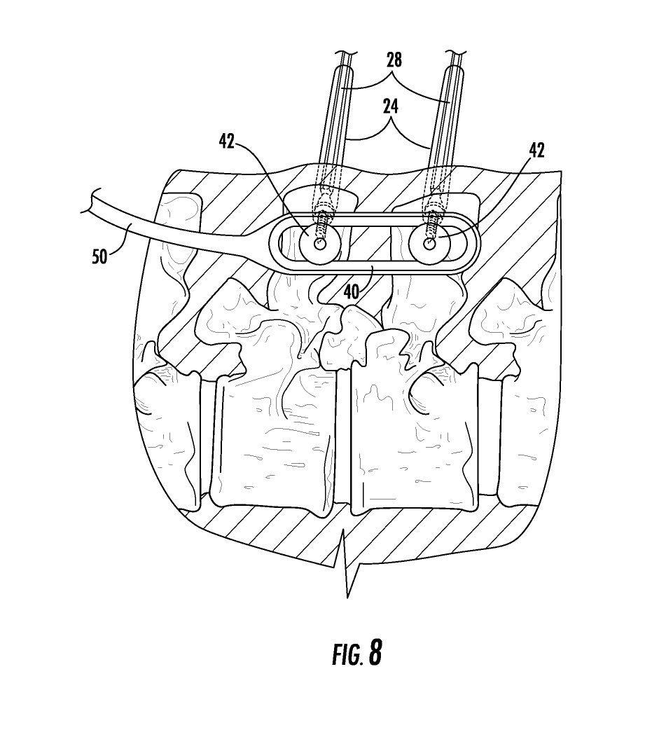

FIG. 6 shows the embodiment of the present invention as seen in FIG. 4, Step 4, with the k-wire 20 and second cannula 24 inserted into the channel in the skin of a patient and directed towards two adjacent bones of the spine prior to being drilled therethrough. However, this image discloses the angle from the side of the spine. The stabilizing plate 40 is preferably inserted by the device and method described above onto the side of the spinous process opposite that on which the k-wire 20 is inserted, and the stabilizing plate 40 is maneuvered to the spinous process by means of carrier or tool 50. This is shown in FIG. 7. Carrier or tool 50 will preferably align the stabilizing plate with the screws 30 or staples as will be drilled through the spinous processes, so that the screws 30 mate with the caps 42 of the stabilizing plate after passage through the bone (or the screws/staples with the nuts). Once connection is made, and the stabilizing plate is securely held in position, carrier 50 can be removed in the same manner it was entered into the body. Of course the tool removably secures the plate, or a mechanism is provided for holding and then releasing the plate as desired. FIG. 8 discloses the Step of the screws being drilled through adjacent bones of the spinous process for attachment to the caps 42 of an elongated stabilizing plate 40. As can be seen, the carrier or tool 50 remains in place until the drilling and attachment of the screws 30 is complete.

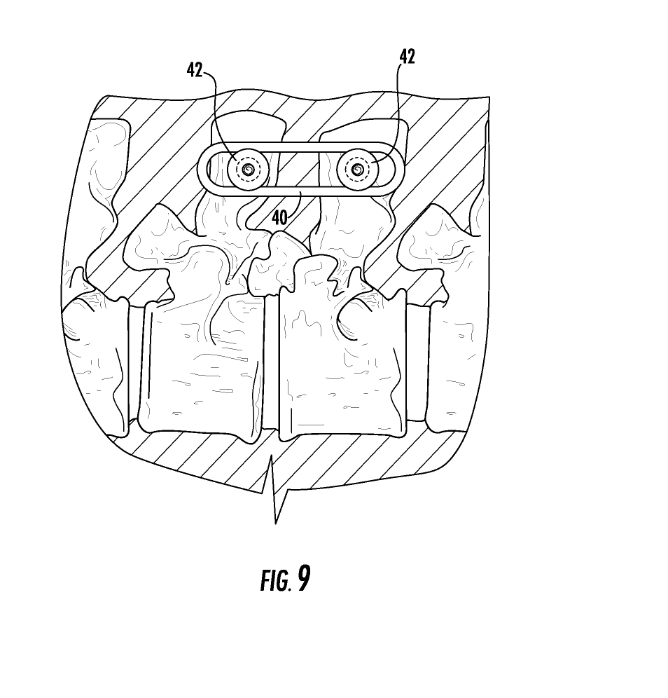

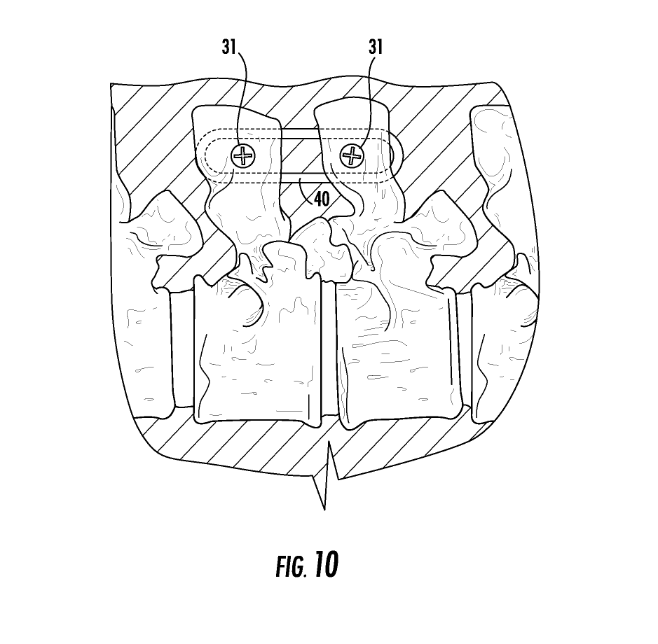

Once such drilling occurs, the drill 28, and second cannula 24 can be removed from the body, leaving behind the stabilizing plate 40 secured to the spinous processes by means of screws 30 attached to caps 42 through the bones. The view from the side of the patient and showing the stabilizing plate secured to two adjacent bones is shown in FIG. 9, while the view of FIG. 10 is with the frills, cannulas, etc., removed, i.e., after the procedure is complete.

It will be understood by those of ordinary skill in the art that various changes may be made and equivalents may be substituted for elements without departing from the scope of the invention. In addition, many modifications may be made to adapt a particular feature or material to the teachings of the invention without departing from the scope thereof. Therefore, it is intended that the invention not be limited to the particular embodiments disclosed, but that the invention will include all embodiments falling within the scope of the claims.

What is claimed:

1. A method for performing percutaneous spinal stabilization, comprising:
inserting a k-wire and drill end into the skin and tissue proximal to a bone in the spine;
inserting one or more cannulas over said k-wire and through the skin and tissue and towards said bone to develop a working channel;
removing said k-wire and said cannulas;
inserting a screwdriver into said working channel, said screwdriver comprising a shaft long enough to extend through said working channel to said bone, and said screwdriver having removably attached, a screw with a turning end and a distal holding end; and
turning said screw to secure said distal end to a stabilizing plate, pre-positioned on a side of said bone.

2. A method as claimed in claim 1, wherein said screw is secured to a cap secured to said stabilizing plate with said screw having external screw threads, mating with internal screw threads of said cap.

3. A method as claimed in claim 1, wherein said stabilizing plate is preferably thin-walled, hollow, oval-shaped and flat.

4. A method as claimed in claim 1, wherein said stabilizing plate has a longitudinal central opening sufficiently long to attach to more than one screw for securement to one or more bones in the spine.

5. A method as claimed in claim 1, further comprising inserting said stabilizing plate into the spine through a delivery tube and by means of a carrier clamp.

6. A method as claimed in claim 5, wherein said carrier clamp which may comprise a pliar-like device which releasably holds the stabilizing plate.

7. A method as claimed in claim 1, wherein the preferred number of cannulas is two, and wherein a first cannula is configured to fit within the longitudinal channel of a wider bore second cannula.

8. A system for performing percutaneous spinal stabilization, comprising:
a k-wire having a leading/drilling point;
one or more cannulas, adapted for insertion through the skin of a patient to create a working channel to the spinous processes by passing over said k-wire;
a stabilizing plate having one or more capturing caps or nuts secured thereto with said caps or nuts having internal screw threads; and
one or more screws with external screw threads mating with said caps or nuts and also having a driving end, adapted to be drilled through a bone of the spine and to securely attach to said caps or nuts of said stabilizing plate.

9. A system as claimed in claim 8, wherein the preferred number of cannulas is two, and wherein a first cannula is configured to fit within the longitudinal channel of a second cannula.

10. A system as claimed in claim 8, wherein said stabilizing plate is preferably with opposing, flat-walls, hollow, and oval-shaped.

11. A method as claimed in claim 8, wherein said stabilizing plate is adapted to be inserted into the spine through a delivery tube and by means of a carrier clamp which releasably secures the same in position.
